(19) United States
(12) Patent Application Publication
(10) Pub. No.: US 2023/0365633 A1
(43) Pub. Date: Nov. 16, 2023

(54) GRIFFITHSIN FOR RHABDOVIRIDAE INFECTIONS

(71) Applicants: The United States of America,as represented by the Secretary,Department of Health and Human Services, Bethesda, MD (US); University of Louisville Research Foundation, Inc., Louisville, KY (US)

(72) Inventors: Nadia F. Gallardo-Romero, Suwanee, GA (US); Barry R. O'Keefe, Frederick, MD (US); Kenneth E. Palmer, Owensboro, KY (US)

(73) Assignees: The United States of America,as represented by the Secretary,Department of Health and Human Services, Bethesda, MD (US); University of Louisville Research Foundation, Inc., Louisville, KY (US)

(21) Appl. No.: 18/030,174

(22) PCT Filed: Oct. 9, 2020
(86) PCT No.: PCT/US2020/054930

§ 371 Date: Apr. 4, 2023

(51) Int. Cl. C07K 14/405; A61K 45/06; A61P 31/14

(52) U.S. Cl. CPC C07K 14/405; A61K 45/06; A61P 31/14

(57) ABSTRACT

The invention is directed to methods of treating or preventing a Rhabdoviridae virus infection in a mammal comprising administering griffithsin, or a fragment or mutant thereof, to the mammal.

### STATEMENT REGARDING FEDERALLY SPONSORED RESEARCH OR DEVELOPMENT

This invention was made with Government support under project number ZIABC011472 by the National Cancer Institute of the National Institutes of Health. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 18,979 Byte ASCII (Text) file named “749421_ST25.txt,” created on Sep. 18, 2020.

### BACKGROUND OF THE INVENTION

Rhabdoviridae are enveloped RNA viruses characterized by their shape. Rabies is a Rhabdoviridae virus infection that affects animals and humans. Rabies infection leads to fatal encephalitis if left untreated. Millions of people survive rabies infection each year due to timely administration of post-exposure treatment, however, more than 70,000 people die each year because they failed to receive such treatment. Obstacles to timely treatment include the high cost and the burdensome storage requirements (e.g., refrigeration) of current post-exposure treatments. Accordingly, new treatments for Rhabdoviridae viruses, including rabies, are needed with increased availability.

### BRIEF SUMMARY OF THE INVENTION

The invention provides methods of treating a Rhabdoviridae virus infection in a mammal comprising administering griffithsin (GRFT), or a fragment or mutant thereof, to the mammal.

The invention further provides methods of preventing a Rhabdoviridae virus infection in a mammal comprising administering GRFT, or a fragment or mutant thereof, to the mammal.

### BRIEF DESCRIPTION OF THE SEVERAL VIEWS OF THE DRAWINGS

FIG. 1 shows the levels of GRFT observed in Syrian hamster brain tissue (squares) and in the control buffered saline (“PBS,” circles).

FIG. 2 is a graph showing the percent survival rate of Syrian hamsters following treatment for rabies virus infection.

### DETAILED DESCRIPTION OF THE INVENTION

The invention provides methods of treating and/or preventing a Rhabdoviridae virus infection in a mammal comprising administering GRFT, or a fragment or mutant thereof, to the mammal. Also provided is a griffithsin polypeptide, or a fragment or mutant thereof, useful for such methods.

### Rhabdoviridae Viruses

Rhabdoviridae viruses are rod- or bullet-shaped and are single-stranded, negative-sense, and nonsegmented RNA viruses. In an embodiment, the Rhabdoviridae virus is a Rhabdoviridae virus that causes an infection or disease in a mammal. In an embodiment, the Rhabdoviridae virus is a Lyssavirus. In an embodiment, the Rhabdoviridae virus is Aravan lyssavirus, Australian bat lyssavirus, Bokeloh bat lyssavirus, Duvenhage lyssavirus, European bat 1 lyssavirus, European bat 2 lyssavirus, Gannoruwa bat lyssavirus, Ikoma lyssavirus, Irkut lyssavirus, Khujand lyssavirus, Lagos bat lyssavirus, Lleida bat lyssavirus, Mokola lyssavirus, rabies virus, Shimoni bat lyssavirus, Taiwan bat lyssavirus, or West Caucasian bat lyssavirus. In an embodiment, the Rhabdoviridae virus is rabies virus.

The rabies virus is an enveloped virus of the Rhabdovirus family and Lyssavirus genus. The genome of rabies virus codes five proteins: RNA-dependent RNA polymerase (L); a nucleoprotein (N); a phosphorylated protein (P); a matrix protein (M) located on the inner side of the viral protein envelope; and an external surface glycoprotein (G). The G protein (62-67 kDa) is a type-I glycoprotein composed of 505 amino acids, with two to four potential N-glycosylation sites, of which only one or two are glycosylated depending on the viral strain. The G protein forms protrusions covering the outer surface of the virion envelope and is known to induce the production virus-neutralizing antibodies.

Rabies infection can be treated or prevented by both passive and active immunizations. Rabies post-exposure prophylaxis (PEP) includes prompt local wound care and administration of both passive (anti-rabies immunoglobulins) and active (vaccines) immunizations. Currently, the anti-rabies immunoglobulins (RIG) are prepared from the serum of either human (HRIG) or equine (ERIG) subjects. The use of immunoglobulins from these sources poses several potential difficulties, however, including risk of disease transmission, cost, and in the case of equine immunoglobulin, adverse reactions such as anaphylactic shock.

### Griffithsin and Fragments, Mutants, and Constructs Thereof

GRFT is a lectin which has been shown to be a potent HIV inhibitor. It has a 121-amino-acid sequence (SEQ ID NO: 2) and was isolated from the red algae Griffithsia. In one embodiment, GRFT is administered to the mammal. In some embodiments, the GRFT, or a fragment thereof, has about 75%, about 80%, about 85%, about 90%, about 91%, about 92%, about 93%, about 94%, about 95%, about 96%, about 97%, about 98%, about 99% or more identity to SEQ ID NO: 2.

GRFT, or a fragment or mutant thereof, may include the lectins disclosed in the following publications: US20090297516A1, WO2016130628A1, US20160108097A1, US20100221242A1, US20110189105A1, US20090092557A1, US20110263485A1, US20100240578A1, the entire disclosures of which are incorporated by reference.

Included in the scope of an embodiment of the invention are GRFT mutants described herein. The phrases “griffithsin mutants,” “GRFT mutants,” “griffithsin, or mutant thereof,” or “GRFT, or mutant thereof” as used herein refer to a lectin having substantial or significant sequence identity or similarity to a GRFT, which mutant retains the biological activity of GRFT. Mutants encompass, for example, mutants of GRFT that retain the ability to treat or prevent Rhabdoviridae virus to a similar extent, the same extent, or to a higher extent, as GRFT. In reference to the GRFT, the mutant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to SEQ ID NO: 2.

A GRFT mutant can, for example, comprise the amino acid sequence of the GRFT with at least one conservative amino acid substitution. Alternatively, or additionally, the functional variants can comprise the amino acid sequence of GRFT with at least one non-conservative amino acid substitution. In this case, it is preferable for the non-conservative amino acid substitution to not interfere with or inhibit the biological activity of the GRFT mutant. The non-conservative amino acid substitution may enhance the biological activity of the GRFT mutant, such that the biological activity of the GRFT mutant is increased as compared to the GRFT.

Amino acid substitutions GRFT mutants are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

GRFT, and mutants thereof, can consist essentially of the specified amino acid sequence or sequences described herein, such that other components, e.g., other amino acids, do not materially change the biological activity of the protein, functional portion, or functional variant. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypiation) or deglycosylated.

GRFT fragments and mutants thereof of embodiments of the invention can be of any length, i.e., can comprise any number of amino acids, provided that the GRFT, or a fragment or mutant thereof, retains their biological activity, e.g., prevent or treat Rhabdoviridae virus infection in a mammal, e.g., a human. For example, GRFT, or a fragment or mutant thereof, can be from about 50 to about 5,000 amino acids long, such as 50, 70, 75, 100, 125, 150, 175, 200, 300, 400, 500, 600, 700, 800, 900, 1,000, or more amino acids in length.

GRFT, or a fragment or mutant thereof, of embodiments of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,β-diaminopropionic acid, homophenylalanine, and α-tert-butylglycine.

In an embodiment, the GRFT mutant comprises, consists essentially of, or consists of SLTHRKFGGSGGSPFSGLSSIAVRSGSYLDAIIIDGVHHGGSGGNLSPTFTFGSGEYISN X₁TIRSGDYIDNISFX₂TNX₃GRRFGPYGGSGGSANTLSNVKVIQINGX₄X₅GDYLDSLD X₆YYX₇QY (SEQ ID NO: 1), wherein X₁ can be M or V, X₂ can be E or Q, X₃ can be M, A, K, V, F, L, I, Q, R, or G, X₄ can be S or R, X₅ can be A or S, X₆ can be I or F, and X₇ can be E or Q provided that the polypeptide does not comprise the amino acid sequence of SEQ ID NO: 2 (the polypeptide encoded by SEQ ID NO: 2 is referred to herein as “wild type GRFT”).

In an embodiment, the GRFT mutant (or a fragment thereof) comprises a mutation at position 78 (e.g., a Met-Ala mutation) relative to the wild-type GRFT sequence (SEQ ID NO: 2). The GRFT mutant can contain an amino acid at position 78 relative to the wild-type GRFT sequence (SEQ ID NO: 2) that is not charged and contains no sulfur. Exemplary amino acids include Ala, Lys, Val, Gly, Leu, Ile, and Phe. In one embodiment, the GRFT mutant polypeptide comprises, consists essentially of, or consists of the amino acid sequence of SEQ ID NO: 11. In other embodiments, the griffithsin mutant polypeptide comprises, consists essentially of, or consists of the amino acid sequence of SEQ ID NO: 12 or 13.

Alternatively, GRFT fragments or mutants can contain an amino acid at position 78 relative to the wild-type GRFT sequence (SEQ ID NO: 2) that is charged, such as a basic amino acid (e.g., Glu). In one embodiment, the GRFT mutant polypeptide comprises, consists essentially of, or consists of the amino acid sequence of SEQ ID NO: 14 (the polypeptide encoded by SEQ ID NO: 14 is referred to herein as “Q-GRFT”). In one embodiment, the GRFT mutant can be at least about 30%, about 50%, about 75%, about 80%, about 85%, about 90%, about 91%, about 92%, about 93%, about 94%, about 95%, about 96%, about 97%, about 98%, about 99% or more identical to amino acid sequence to SEQ ID NO: 14.

The GRFT fragments or mutants also can contain mutations that change the isoelectric point of the protein (e.g., at positions 75, 78, and 119) and alter its solubility in various pH ranges (e.g., at positions 106 and 107). Additionally, the GRFT mutants can contain mutations at positions 61 and 116, which are related to methionine oxidation.

In an embodiment, the GRFT mutant polypeptide comprises, consists essentially of, or consists of the amino acid sequence of SEQ ID NO: 3 (corresponding to the dVQK mutant). The amino acid sequence of SEQ ID NO: 3 corresponds to the amino acid sequence of SEQ ID NO: 1, wherein X₁, X₂, X₃, X₄, X₅, X₆, and X₇ of SEQ ID NO: 1 are V, Q, K, S, A, I, and E, respectively.

In an embodiment, the GRFT mutant polypeptide comprises, consists essentially of, or consists of the amino acid sequence of SEQ ID NO: 4 (corresponding to the dVQKR mutant). The amino acid sequence of SEQ ID NO: 4 corresponds to the amino acid sequence of SEQ ID NO: 1, wherein X₁, X₂, X₃, X₄, X₅, X₆, and X₇ of SEQ ID NO: 1 are V, Q, K, R, A, I, and E, respectively.

In an embodiment, the GRFT mutant polypeptide comprises, consists essentially of, or consists of the amino acid sequence of SEQ ID NO: 5 (corresponding to the dVQKFQ mutant). The amino acid sequence of SEQ ID NO: 5 corresponds to the amino acid sequence of SEQ ID NO: 1, wherein X₁, X₂, X₃, X₄, X₅, X₆, and X₇ of SEQ ID NO: 1 are V, Q, K, S, A, F, and Q, respectively.

In an embodiment, the GRFT mutant polypeptide comprises, consists essentially of, or consists of the amino acid sequence of SEQ ID NO: 6 (corresponding to the dQKR mutant). The amino acid sequence of SEQ ID NO: 6 corresponds to the amino acid sequence of SEQ ID NO: 1, wherein X₁, X₂, X₃, X₄, X₅, X₆, and X₇ of SEQ ID NO: 1 are M, Q, K, R, A, I, and E, respectively.

In additional embodiments, the GRFT mutant polypeptide comprises, consists essentially of, or consists of:
(i) the amino acid sequence of SEQ ID NO: 1, wherein X₁, X₂, X₃, X₄, X₅, X₆, and X₇ are M, Q, K/V/A, S, A, I, and E, respectively (corresponding to SEQ ID NO: 7);
(ii) the amino acid sequence of SEQ ID NO: 1, wherein X₁, X₂, X₃, X₄, X₅, X₆, and X₇ are M, Q, K, S, A, I, and Q, respectively (corresponding to SEQ ID NO: 8);
(iii) the amino acid sequence of SEQ ID NO: 1, wherein X₁, X₂, X₃, X₄, X₅, X₆, and X₇ are V, Q, K, S, A, I and Q, respectively (corresponding to SEQ ID NO: 9); or
(iv) the amino acid sequence of SEQ ID NO: 1, wherein X₁, X₂, X₃, X₄, X₅, X₆, and X₇ are M, Q, K, S, A, F, and Q, respectively (corresponding to SEQ ID NO: 10).

The sequences and mutants of GRFT mutant polypeptides are set forth in Table 1 below.

TABLE 1

| Mutations | SEQ ID NO |
|---|---|
| None (wild-type) | 2 |
| M61V, E75Q, M78K | 3 |
| M61V, E75Q, M78K, S106R | 4 |
| M61V, E75Q, M78K, I116F, E119Q | 5 |
| E75Q, M78K, S106R | 6 |
| E75Q, M78K/V/A | 7 |
| E75Q, M78K, E119Q | 8 |
| E75Q, M61V, M78K, E119Q | 9 |
| E75Q, M61V, M78K, I116F, E119Q | 10 |

If desired, the GRFT mutant polypeptides of the invention (including antiviral fragments, fusion proteins, constructs, and conjugates) can be modified, for instance, by glycosylation, amidation, carboxylation, or phosphorylation, or by the creation of acid addition salts, amides, esters, in particular C-terminal esters, and N-acyl derivatives of the proteins of the invention. The polypeptides also can be modified to create protein derivatives by forming covalent or noncovalent complexes with other moieties in accordance with methods known in the art. Covalently-bound complexes can be prepared by linking the chemical moieties to functional groups on the side chains of amino acids comprising the proteins, or at the N- or C-terminus. Desirably, such modifications and conjugations do not adversely affect the activity of the polypeptides. While such modifications and conjugations can have greater or lesser activity, the activity desirably is not negated and is characteristic of the unaltered polypeptide.

The GRFT mutant polypeptides can contain additional insertions, deletions, substitutions, or additions. However, in a preferred embodiment, the GRFT mutant polypeptides form dimers like wild-type GRFT. In other words, the changes to the wild-type GRFT amino acid sequence do not result in the loss of the ability of the GRFT mutant polypeptides to form dimers (i.e., monomeric GRFT). GRFT dimers have been reported to have increased potency (e.g., about 1000 times increased potency) when compared to GRFT monomers (Moulaei et al., Structure, 18(9): 1104-1115 (2010)). Therefore, in a preferred embodiment, the GRFT mutant polypeptides do not contain a substitution at Leu2 (e.g., Leu2Ser) relative to the amino acid sequence of GRFT (SEQ ID NO: 2) and/or an insertion of two or more residues between Ser16 and Gly17 (e.g., (Gly-Ser)ₙ, wherein n is 1 or 2) relative to the amino acid sequence of GRFT (SEQ ID NO: 2) without compensating mutations/insertions that would allow for multimeric versions of GRFT monomers in sequence (i.e., GRFT tandemers). Although not wishing to be bound by any particular theory, the L2S and (Gly-Ser)ₙ mutations are believed to be related to obligate monomeric GRFT structures. Additionally or alternatively, the GRFT mutant polypeptides can include N-terminal modifications, such as N-aceytlation (e.g., an N-terminal serine acetylated on the amino group). N-acetylation increases stability to aminopeptidases (O'Keefe et al., PNAS, 106(15): 6099-6104 (2009)).

The polypeptides (and fragments, fusion proteins, and constructs) can be prepared by any of a number of conventional techniques. The polypeptide can be isolated or purified from a recombinant source. For instance, a DNA fragment encoding a desired polypeptide can be subcloned into an appropriate vector using well-known molecular genetic techniques. The fragment can be transcribed and the polypeptide subsequently translated in vitro. Commercially available kits also can be employed. The polymerase chain reaction optionally can be employed in the manipulation of nucleic acids.

Such polypeptides also can be synthesized using an automated peptide synthesizer in accordance with methods known in the art. Alternately, the polypeptide (and fragments, fusion proteins, and constructs) can be synthesized using standard peptide synthesizing techniques well-known to those of skill in the art (e.g., as summarized in Bodanszky, Principles of Peptide Synthesis, (Springer-Verlag, Heidelberg: 1984)). In particular, the polypeptide can be synthesized using the procedure of solid-phase synthesis (see, e.g., Merrifield, J Am. Chem. Soc., 85: 2149-54 (1963); Barany et al., Int. J. Peptide Protein Res., 30: 705-739 (1987); and U.S. Pat. No. 5,424,398, incorporated herein by reference). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The protein-containing mixture then can be extracted, for instance, with diethyl ether, to remove non-peptidic organic compounds, and the synthesized polypeptide can be extracted from the resin powder (e.g., with about 25% w/v acetic acid). Following the synthesis of the polypeptide, further purification (e.g., using HPLC) optionally can be performed in order to eliminate any incomplete proteins, polypeptides, peptides or free amino acids. Amino acid and/or HPLC analysis can be performed on the synthesized polypeptide to validate its identity.

For other applications according to the invention, it may be preferable to produce the GRFT or mutant GRFT polypeptide (or a fragment thereof) as part of a larger fusion protein, either by chemical conjugation or through genetic means, such as are known to those skilled in the art. In this regard, the invention also provides a fusion protein comprising the GRFT or GRFT mutant polypeptide (or a fragment thereof) and one or more other protein(s) having any desired properties or effector functions, such as cytotoxic or immunological properties, or other desired properties, such as to facilitate isolation, purification, analysis, or stability of the fusion protein.

The GRFT or mutant thereof (or a fragment thereof) also can be part of a conjugate. For instance, the GRFT or mutant can be coupled to at least one effector component, or multiple effector components which can be the same or different. The effector component can be polyethylene glycol, dextran, albumin, an immunological reagent, a toxin, an antiviral agent, or a solid support matrix. “Immunological reagent” includes, but is not limited to, an antibody, an antibody fragment (e.g., an F(ab′)2, an Fab′, an Fab, an Fv, an scFv, a dsFv, an eAd, or an Fc antibody fragment), an immunoglobulin, and an immunological recognition element. An immunological recognition element is an element, such as a peptide, e.g., the FLAG sequence of a recombinant GRFT or GRFT mutant polypeptide-FLAG fusion protein, which facilitates, through immunological recognition, isolation and/or purification and/or analysis of the protein or peptide to which it is attached. An immunological reagent also can be an immunogenic peptide, which can be fused to the GRFT or GRFT mutant polypeptide for enhancing an immune response.

In this respect, the invention provides an antiviral conjugate comprising GRFT, or a fragment or mutant thereof, bound to a virus or viral envelope glycoprotein. The GRFT, or a fragment or mutant thereof, fusion protein is a type of GRFT, or a fragment or mutant thereof, polypeptide conjugate, wherein the GRFT, or a fragment or mutant thereof, polypeptide is coupled to one or more other protein(s) having any desired properties or effector functions, such as cytotoxic or immunological properties, or other desired properties, such as to facilitate isolation, purification or analysis of the fusion protein or increase the stability or in vivo half-life of the fusion protein. The GRFT, or a fragment or mutant thereof, polypeptide also can be attached to a chemical moiety which allows recognition, isolation, purification, and/or analysis of the protein or peptide. An example of such a chemical moiety is a His tag.

A “toxin” can be, for example, Pseudomonas exotoxin. An “antiviral agent” can be AZT, ddI, ddC, 3TC gancyclovir, fluorinated dideoxynucleosides, nevirapine, R82913, Ro 31-8959, BI-RJ-70, acyclovir, α-interferon, recombinant sCD4, michellamines, calanolides, nonoxynol-9, gossypol and derivatives thereof, gramicidin, amantatadine, rimantadine, and neuraminidase inhibitors, cyanovirin-N or a functional homolog or derivative thereof (see, for example, U.S. Pat. No. 5,843,882, incorporated herein by reference), or scytovirin or a functional homolog or derivative thereof (see, e.g., U.S. Pat. Nos. 7,494,798 and 8,067,530, incorporated herein by reference). A “solid support matrix” can be a magnetic bead, a flow-through matrix, a sponge, a stent, a culture plate, or a matrix comprising a contraceptive device, such as a condom, diaphragm, cervical cap, vaginal ring or contraceptive sponge. In an alternative embodiment, a solid support matrix can be an implant for surgical implantation in a host and, if appropriate, later removal.

Conjugates furthermore can comprise the GRFT or mutant GRFT polypeptide (or a fragment thereof) polypeptides coupled to more than one effector molecule, each of which, optionally, can have different effector functions (e.g., such as a toxin molecule (or an immunological reagent) and a polyethylene glycol (or dextran or albumin) molecule). Diverse applications and uses of functional proteins and peptides attached to or immobilized on a solid support matrix, are exemplified more specifically for poly (ethylene glycol) conjugated proteins or peptides in a review by Holmberg et al. (In Poly(Ethylene Glycol) Chemistry: Biotechnical and Biomedical Applications, Harris, ed., Plenum Press, New York (1992), pp. 303-324).

GRFT constructs refer to constructs comprising two or more GRFT molecules, or fragments or mutants thereof, joined by a linker. Suitable linkers include linkers that are at least about 3 (e.g., 3, 4, 5, 6, 7, 8, 9, 10, 11, or 12) amino acids in length. Examples of suitable linkers include, but are not limited to, linkers that comprise one or more (e.g., two, three, four, five, six, seven, eight, nine, or ten) (Gly-Thr-Gly) n motifs (wherein n is 1-5).

Using an appropriate nucleic acid (e.g., DNA) coding sequence, the GRFT or GRFT mutant polypeptides (or a fragment thereof), fusion proteins, constructs, and conjugates can be made by genetic engineering techniques (for general background see, e.g., Nicholl, in An Introduction to Genetic Engineering, Cambridge University Press: Cambridge (1994), pp. 1-5 & 127-130; Steinberg et al., in Recombinant DNA Technology Concepts and Biomedical Applications, Prentice Hall: Englewood Cliffs, NJ (1993), pp. 81-124 & 150-162; Sofer in Introduction to Genetic Engineering, Butterworth-Heinemann, Stoneham, MA (1991), pp. 1-21 & 103-126; Old et al., in Principles of Gene Manipulation, Blackwell Scientific Publishers: London (1992), pp. 1-13 & 108-221; and Emtage, in Delivery Systems for Peptide Drugs, Davis et al., eds., Plenum Press: New York (1986), pp. 23-33). For example, DNA encoding the GRFT or mutant GRFT polypeptide (or a fragment thereof) polypeptides, fusion proteins, constructs, and conjugates can be incorporated into an appropriate expression vector and delivered into an appropriate polypeptide-synthesizing organism (e.g., E. coli, S. cerevisiae, P. pastoris, or other bacterial, yeast, insect, plant or mammalian cells), where the DNA, under the control of an endogenous or exogenous promoter, can be appropriately transcribed and translated. Alternatively, the expression vector can be administered to a plant or animal, for example, for large-scale production (see, e.g., Fischer et al., Transgenic Res., 9(4-5): 279-299 (2000); Fischer et al., J Biol. Regul. Homeost. Agents, 14: 83-92 (2000); deWilde et al., Plant Molec. Biol., 43: 347-359 (2000); Houdebine, Transgenic Research, 9: 305-320 (2000); Brink et al., Theriogenology, 53: 139-148 (2000); Pollock et al., J Immunol. Methods, 231: 147-157 (1999); Conrad et al., Plant Molec. Biol., 38: 101-109 (1998); Staub et al., Nature Biotech., 18: 333-338 (2000); McCormick et al., PNAS USA, 96: 703-708 (1999); Zeitlin et al., Nature Biotech., 16: 1361-1364 (1998); Tacker et al., Microbes and Infection, 1: 777-783 (1999); Tacket et al., Nature Med., 4(5): 607-609 (1998); and Methods in Biotechnology, Recombinant Proteins from Plants, Production and Isolation of Clinically Useful Compounds, Cunningham and Porter, eds., Humana Press: Totowa, New Jersey (1998)). Such expression vectors (including, but not limited to, phage, cosmid, viral, and plasmid vectors) are known to those skilled in the art, as are reagents and techniques appropriate for gene transfer (e.g., transfection, electroporation, transduction, micro-injection, transformation, etc.). If the GRFT or GRFT mutant polypeptides (or fragments thereof) are to be recombinantly produced in isolated eukaryotic cells or in a eukaryotic organism, such as a plant (see above references and also Methods in Biotechnology, Recombinant Proteins from Plants, Production and Isolation of Clinically Useful Compounds, Cunningham and Porter, eds., Humana Press: Totowa, New Jersey (1998)), any glycosylation sites in the polypeptides are rendered glycosylation resistant (e.g., the N-linked glycosylation sites at positions 45, 60, 71, and/or 104 relative to the amino acid sequence of GRFT (SEQ ID NO: 2)). Subsequently, the recombinantly produced polypeptide can be isolated and purified using standard techniques known in the art (e.g., chromatography, centrifugation, differential solubility, isoelectric focusing, etc.), and assayed for antiviral activity.

### Routes of Administration, Dosing, and Duration

GRFT, or a fragment or mutant thereof, can be administered to a mammal using any suitable administration techniques, many of which are known in the art, including oral, intravenous, intraperitoneal, subcutaneous, pulmonary, transdermal, intramuscular, intranasal, buccal, sublingual, or suppository administration. GRFT, or a fragment or mutant thereof, is suitable for parenteral administration. The term “parenteral,” as used herein, includes intravenous, intramuscular, subcutaneous, rectal, vaginal, and intraperitoneal administration. More preferably, GRFT, or a fragment or mutant thereof, is administered to a mammal using intramuscular injection (IM), oral, or subcutaneous routes.

When the inventive method comprises administering GRFT, or a mutant thereof, to a mammal, the GRFT, or a mutant thereof, is administered to the mammal at a dose sufficient to induce the treatment or prevention of Rhabdoviridae virus in the mammal. Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of GRFT, or a fragment or mutant thereof, by multiple bolus administrations of GRFT, or a fragment or mutant thereof, or by continuous infusion administration of GRFT, or mutant thereof.

In an embodiment, the method comprises administering GRFT, or a fragment or mutant thereof, to the mammal in a single dose. In another embodiment, the method comprises administering GRFT, or a fragment or mutant thereof, to the mammal in multiple doses. In another embodiment, the method comprises administering GRFT, or a fragment or mutant thereof, to the mammal in two to five separate doses with a dosing interval of one to five days (i.e., one, two, three, four, or five days).

GRFT, or a fragment or mutant thereof, can be administered to a mammal over any suitable duration. For example, GRFT, or a fragment or mutant thereof, can be administered to the mammal for at least a 30 day duration (e.g., about 30 days, about 35 days, about 40 days, about 45 days, about 50 days, about 55 days, about 60 days, about 65 days, about 70 days), or during an alternative duration necessary to complete the treatment or prevention of Rhabdoviridae virus in the mammal.

### Additional Therapeutic Agent

GRFT, or a fragment or mutant thereof, can be co-administered with an additional therapeutic agent. The additional therapeutic agent can be any suitable therapeutic agent including rabies immune globulin (RIG), a rabies vaccine, an antibody (e.g., a humanized monoclonal antibody) or a fragment thereof that binds to an epitope on a rabies virus glycoprotein, an antiviral medication, a single stranded RNA with immunostimulating activity, or a combination thereof.

In an embodiment, the additional therapeutic agent is a humanized monoclonal antibody that binds to an epitope on a rabies virus glycoprotein. Any suitable humanized monoclonal antibody that binds to an epitope on a rabies virus glycoprotein can be administered, for example, antibodies CTB011, CTB012, RAB1, CR57, CR4098, or a combination thereof. Alternatively, an antibody or antibody fragment containing the Complementarity-Determining Regions (CDRs) and/or variable regions of CTB011, CTB012, RAB1, CR57, or CR4098 can be administered.

CTB011 and CTB012 are described in Chao et al., PLoS Negl TropDis., 11(12): e0006133 (2017), RAB1 is available from MassBiologics and Serum Institute of India, Pvt. Ltd., and CR57 and CR4098 can be mixed as a cocktail (e.g., in a 1:1 protein ratio) and co-administered (available as “CL184” from Crucell, see Goudsmit et al., The Journal of Infectious Diseases, 193(6): 796-801 (2006). In an embodiment, the additional therapeutic agent is a mixture of CTB011 and CTB012. In an embodiment, the additional therapeutic agent is a mixture of CR57 and CR4098.

In one embodiment, the antibody fragment is a Fab fragment (Fab), F(ab′)2 fragment, diabody, triabody, tetrabody, bispecific antibody, single-chain variable region fragment (scFv), or disulfide-stabilized variable region fragment (dsFv).

The antibody can be of any isotype (e.g., isotype IgA, IgD, IgE, IgG (e.g., IgG1, IgG2, IgG3, IgG4, or IgM). In one embodiment, the antibody is a naturally-occurring antibody, e.g., an antibody isolated and/or purified from a mammal, e.g., mouse, rabbit, goat, horse, chicken, hamster, human, etc. In another embodiment, the antibody is a genetically-engineered antibody, e.g., a humanized antibody or a chimeric antibody. The antibody can be in monomeric or polymeric form. Also, the antibody can have any level of affinity or avidity for rabies virus.

Methods of testing antibodies for the ability to bind to rabies virus are known in the art and include any antibody-antigen binding assay, such as, for example, radioimmunoassay (“RIA”), enzyme-linked immunosorbent assay (“ELISA”), Western blot, immunoprecipitation, and competitive inhibition assays (see, e.g., Murphy et al., infra, and U.S. Patent Application Publication No. 2002/0197266 A1).

In an embodiment, the single stranded RNA (ssRNA) with immunostimulating activity is CV8102. CV8102 is a 547 nucleotide, noncoding, uncapped ssRNA containing several polyU-repeats complexed with a polymeric carrier formed by disulfide-crosslinked cationic peptides. CV8102 is available from CureVac AG, Tübingen, Germany.

In an embodiment, the additional therapy is an antiviral medication. Any suitable antiviral medication can be used. Suitable antiviral medications include ribavirin, also known as tribavirin and 1-(D-D-ribofuranosyl)-1H-1,2,4-triazole-3-carboxamide; amantadine, also known as 1-adamantylamine; and PAV-866, also known as N-(7-(1,4-diazepan-1-yl)-1,9-diethyl-3H-phenothiazin-3-ylidene)-N-methylmethanaminium chloride (available from Prosetta, San Francisco, CA). PAV-866 is an antiviral compound that interrupts host-rabies virus protein-protein interactions.

In an embodiment, the additional therapeutic agent is rabies immune globulin (or immunoglobulin) (RIG). As noted above, RIG is a mixture of anti-rabies immunoglobulins that are prepared from the serum of either human (HRIG) or equine (ERIG) subjects.

In an embodiment, the additional therapeutic agent is a rabies vaccine. Any suitable rabies vaccine can be used. Rabies vaccines are commercially available (e.g., RABAVER™, RABIPUR™, and RABIVAX™).

The terms “treat,” and “prevent” as well as words stemming therefrom, as used herein, do not necessarily imply 100% or complete treatment or prevention of a Rhabdoviridae virus (e.g., rabies). Rather, there are varying degrees of treatment or prevention of which one of ordinary skill in the art recognizes as having a potential benefit or therapeutic effect. In this respect, the inventive methods can provide any amount of any level of treatment or prevention of rabies virus in a mammal (e.g., human). Furthermore, the treatment or prevention provided by the inventive method can include treatment or prevention of one or more conditions or symptoms of a Rhabdoviridae virus (e.g., rabies). Also, for purposes herein, “prevention” can encompass preventing the recurrence of a Rhabdoviridae virus (e.g., rabies), delaying the onset of a Rhabdoviridae virus (e.g., rabies), or a symptom or condition thereof.

GRFT, or a fragment or mutant thereof, can be administered to any mammal in need of treatment or prevention of a Rhabdoviridae virus (e.g., rabies). In an embodiment of the invention, the mammal is a hamster, raccoon, guinea pig, gerbil, squirrel, chipmunk, rat, groundhog, coyote, wolf, mouse, skunk, fox, cat, dog, ferret, mongoose, cow, rabbit, goat, horse, chicken, bat, monkey, ape, or human. In a preferred embodiment, the mammal is a human.

In an embodiment, GRFT, or a fragment or mutant thereof, crosses the blood-brain barrier of the mammal.

In an embodiment, GRFT, or a fragment or mutant thereof, is administered to the mammal before onset of neurological signs of a Rhabdoviridae (e.g., rabies) virus infection.

In an embodiment, GRFT, or a fragment or mutant thereof, binds with a glycoprotein of the Rhabdoviridae (e.g., rabies) viral envelope.

Embodiments of the invention may be beneficial alone or in combination, with one or more other embodiments. Without limiting the foregoing description, certain non-limiting embodiments of the invention are provided below as embodiments numbered 1-27. As will be apparent to those of skill in the art upon reading this disclosure, each of the individually numbered embodiments may be used or combined with any of the preceding or following individually numbered embodiments. As such, the invention provides for all combinations of these embodiments and is not limited to combinations of embodiments explicitly provided below.
1. A method of treating a Rhabdoviridae virus infection in a mammal comprising administering griffithsin, or a fragment or mutant thereof, to the mammal.
2. A method of preventing a Rhabdoviridae virus infection in a mammal comprising administering griffithsin, or a fragment or mutant thereof, to the mammal.
3. The method of embodiment 1 or 2, wherein the griffithsin mutant comprises SLTHRKFGGSGGSPFSGLSSIAVRSGSYLDAIIIDGVHHGGSGGNLSPTFTFGSGEYISN X₁TIRSGDYIDNISFX₂TNX₃GRRFGPYGGSGGSANTLSNVKVIQINGX₄X₅GDYLDSLD X₆YYX₇QY (SEQ ID NO: 1), wherein X₁ can be M or V, X₂ can be E or Q, X₃ can be M, A, K, V, F, L, I, Q, R, or G, X₄ can be S or R, X₅ can be A or S, X₆ can be I or F, and X₇ can be E or Q provided that the polypeptide does not comprise the amino acid sequence of SEQ ID NO: 2.
4. The method of any one of embodiments 1-3, wherein the griffithsin mutant comprises SEQ ID NO: 14.
5. The method of any one of embodiments 1-4, wherein griffithsin, or a fragment or mutant thereof, is administered as an intra-muscular injection.
6. The method of any one of embodiments 1-4, wherein griffithsin, or a fragment or mutant thereof, is administered orally.
7. The method of any one of embodiments 1-4, wherein griffithsin, or a fragment or mutant thereof, is administered subcutaneously.
8. The method of any one of embodiments 1-7, wherein the method comprises administering griffithsin, or a fragment or mutant thereof, to the mammal in a single dose.
9. The method of any one of embodiments 1-8, wherein the method comprises administering griffithsin, or a fragment or mutant thereof, to the mammal in multiple doses.
10. The method of any one of embodiments 1-9, wherein the method comprises administering griffithsin, or a fragment or mutant thereof, to the mammal in two to five separate doses with a dosing interval of one to five days.
11. The method of any one of embodiments 1-9, wherein the method comprises administering griffithsin, or a fragment or mutant thereof, to the mammal every one to five days for a 30 day duration.
12. The method of any one of embodiments 1-11, wherein griffithsin, or a fragment or mutant thereof, is co-administered with an additional therapeutic agent.
13. The method of any one of embodiments 1-12, wherein the Rhabdoviridae virus is a Lyssavirus.
14. The method of any one of embodiments 1-13, wherein the Rhabdoviridae virus is a rabies virus.
15. The method of embodiment 14, wherein the additional therapeutic agent is rabies immune globulin (RIG), a rabies vaccine, a humanized monoclonal antibody that binds to an epitope on a rabies virus glycoprotein, an antiviral medication, a single stranded RNA with immunostimulating activity, an antiviral medication, or a combination thereof.
16. The method of embodiment 15, wherein the monoclonal antibody is CTB011, CTB012, RAB1, CR57, CR4098, or a combination thereof.
17. The method of embodiment 15, wherein the monoclonal antibody is a mixture of CTB011 and CTB012.
18. The method of embodiment 15, wherein the single stranded RNA with immunostimulating activity is CV8102.
19. The method of embodiment 15, wherein the additional therapeutic agent is rabies immunoglobulin (RIG).
20. The method of embodiment 15, wherein the additional therapeutic agent is a rabies vaccine.
21. The method of embodiment 15, wherein the additional therapeutic agent is PAV-866.
22. The method of any one of embodiments 1-21, wherein griffithsin, or a fragment or mutant thereof, is administered to the mammal before onset of neurological signs of rabies virus infection.
23. The method of any one of embodiments 1-22, wherein the mammal is a human.
24. A griffithsin polypeptide, or a fragment or mutant thereof, for treating or preventing a Rhabdoviridae virus infection in a mammal, optionally in accordance with the method of any of embodiments 1-23.
25. Use of a griffithsin polypeptide, or a fragment or mutant thereof, for the preparation of a medicament for the prevention or treatment of a Rhabdoviridae virus infection in a mammal, optionally in accordance with the method of any of embodiments 1-23.
26. Use of a griffithsin polypeptide, or a fragment or mutant thereof, for the preparation of a medicament for the treatment of a Rhabdoviridae virus infection in a mammal.
27. Use of a griffithsin polypeptide, or a fragment or mutant thereof, for the preparation of a medicament for the prevention of a Rhabdoviridae virus infection in a mammal.

The following examples further illustrate the invention but, of course, should not be construed as in any way limiting its scope.

### Example 1

This example demonstrates successful in vitro neutralization activity of GRFT on rabies virus (RABV).

In this study, GRFT's rabies virus neutralization activity was evaluated in vitro using a modified Rapid Fluorescent Foci Inhibition Test (RFFIT, see e.g., Yager et al., The Rapid Fluorescent Focus Inhibition Test (Chapter 17), in Current Laboratory Techniques in Rabies Diagnosis, Research and Prevention, (2): 199-215 (2015)).

Total neutralization occurred with the most concentrated sample (lmg/mL) and with 1:5 (200 μg/ml), 1:25 (40 μg/ml), and 1:125 (8 μg/ml) dilutions.

### Example 2

This example demonstrates the safety of administering a wild type GRFT via intramuscular injection (IM).

Five Syrian hamsters were administered GRFT via IM at a dose of 5 mg/kg (about 100 μL) every 48 hours for 5 doses (D0, D2, D4, D6, D8). Plasma and brain tissues were collected after final dose (D8) at 2 hours, 8 hours, 24 hours, 32 hours, and 48 hours (see Table 3).

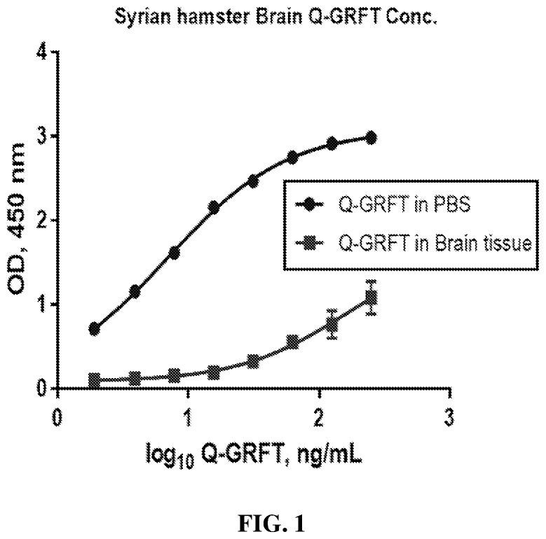

No adverse effects were observed. Surprisingly, GRFT was detected in the brain of the hamsters at concentrations of 6.6 ng/ml per milligram of tissue, 24 hours post treatment, which suggests that griffithsin passed the blood-brain barrier (see FIG. 1 and Tables 2 and 3). The EC₅₀ value refers to the effective concentration of the protein at which 50% of the cells are protected. LLOD refers to the lower level of detection and LLOQ refers to the lower level of quantification. The slight increase in GRFT concentration at 48 hours (versus 32 hours) shown in Table 3 may have been due to blood contamination of the brain sample.

TABLE 2

| | Q-GRFT spiked conc./mg |
|---|---|
| | of naïve brain (ng/ml) |
| EC₅₀ | 159.8 |
| LLOD | 0.327 |
| LLOQ | 0.496 |

TABLE 3

| Time (hours) | GRFT conc/mg of brain sample (ng/ml) |
|---|---|
| 2 | 2.89 |
| 8 | 2.91 |
| 24 | 6.64 |
| 32 | 3.11 |
| 48 | 3.30 |

### Example 3

This example demonstrates the efficacy of administration of GRFT.

GRFT was administered to Syrian hamsters at doses of 5 (“low dose”) and 10 mg/kg (“high dose”) with and without rabies vaccine administration. The rabies vaccines and GRFT were administered 4 days post inoculation with the rabies virus.

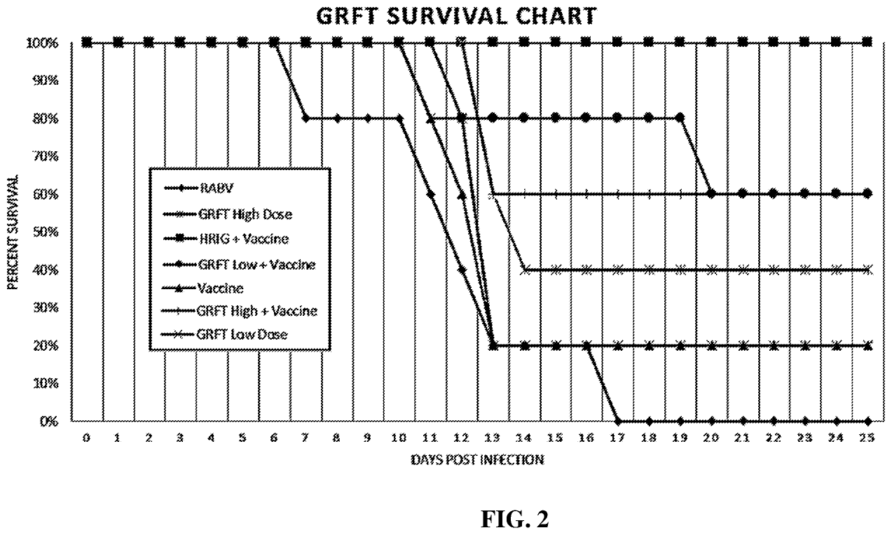

GRFT (high or low dose) when used in combination with vaccine, resulted in 60 survivorship compared to just 20% for the vaccine only against a lethal rabies challenge (see FIG. 2). RIG combined with vaccine resulted in 100 survivorship. Rabies virus inoculation without treatment resulted in 10000 mortality, the low dose of GRFT by itself resulted in a 20% survival rate, and the high dose of GRFT by itself resulted in a 40% survival rate.

TABLE 4

| Group | D0 | D2 | D4 | D6 | D8 | D15 | D21 | D29 |
|---|---|---|---|---|---|---|---|---|
| RABV Only | RABV | X | X | X | X | X | Blood | X |
| RABV + HRIG + | RABV | X | Vaccine | X | Vaccine | Vaccine | Collection | Vaccine |
| Vaccine | HRIG | | | | | | | |
| RABV + Vaccine | RABV | X | Vaccine | X | Vaccine | Vaccine | | Vaccine |
| RABV + GRFT | RABV | GRFT | GRFT | GRFT | GRFT | X | | X |
| Low Dose | GRFT | | | | | | | |
| RABV + GRFT | RABV | GRFT | GRFT | GRFT | GRFT | X | | X |
| High Dose | GRFT | | | | | | | |
| RABV + GRFT | RABV | GRFT | GRFT | GRFT | Vaccine | Vaccine | | Vaccine |
| Low Dose + | GRFT | | Vaccine | | | | | |
| Vaccine | | | | | | | | |
| RABV + GRFT | RABV | GRFT | GRFT | GRFT | Vaccine | Vaccine | | Vaccine |
| High Dose + | GRFT | | Vaccine | | | | | |
| Vaccine | | | | | | | | |

### Example 4

This example further demonstrates the efficacy of administration of GRFT.

GRFT was administered to Syrian hamsters to determine the outcome of pre-exposure and post-exposure treatments. The animals in the Pre-Exposure groups received treatment on day 0 and were challenged against a lethal dose of rabies virus on day 28. All the animals from the Post-Exposure groups were challenged against a lethal dose of rabies virus on day 0 and treated as follows: (a) vaccinated groups received 0.25 IU via IM route on days 0, 3, 7 and 14; and (b) GRFT groups received 20 mg/kg via IM route on days 0, 3, 7 and 14 (or only on day 0 in the GRFT one dose group).

For the pre-exposure treatment group, the animals received either (a) one dose of vaccine and one dose of GRFT (20 mg/kg via IM) 28 days before challenge or (b) one dose of vaccine. Both groups had 100% survival rate. For the post-exposure treatment group, the survival rates were as follows:
Rabies control group had 0% survivors;
Rabies+GRFT only (4 doses) had 50% survivors;
Rabies+vaccine+GRFT (1 dose) had 100% survivors;
Rabies+vaccine+GRFT (4 doses) had 80% survivors; and
Rabies+RIG+Vaccine had 100% survivors.

In addition, the RABV neutralizing activity in sera from animals who received GRFT combined with vaccine (pre-exposure group) was significantly higher than untreated animals (up to 331.4 IU/mL in some individuals, compared to 0.1 IU/mL in the HRIG group on day 21 post infection) indicating that GRFT can be used as a vaccine adjuvant (see Table 5. The only survivor from the vaccine only group had 2.67 IU/ml of RNAb on day 39 post infection.

DFA in Table 5 refers the presence of a fluorescently-labeled anti-rabies antibody. Table 5 shows that the survivors from the GRFT (high and low dose) with vaccine (post-exposure group) showed between 66.67 and 331.4 IU/ml of RNAb in serum on day 21 post-infection. By day 45 post-infection, a few animals had increased the antibody response to 1663.7 IU/ml. (RNAb <0.05 IU/ml are considered protective.)

TABLE 5

| Experimental | | | RNAb D21/or | RNAb |
|---|---|---|---|---|
| Group | DFA | RNAb D0 | EU date | D45/survivors |
| RABV + GRFT | POS | <0.05 | <0.05 | NA |
| Low Dose | POS | <0.05 | <0.05 | NA |
| | NEG | <0.05 | 0.1 | 2.67 |
| | POS | <0.05 | <0.05 | NA |
| | POS | <0.05 | <0.05 | NA |
| RABV + GRFT | POS | <0.05 | <0.05 | NA |
| High Dose | NEG | <0.05 | 0.1 | 0.1 |
| | POS | <0.05 | <0.05 | NA |
| | POS | <0.05 | No Serum | NA |
| | NEG | <0.05 | 0.1 | 0.1 |
| RABV + HRIG + | NEG | <0.05 | 0.1 | 0.5 |
| Vaccine | NEG | <0.05 | 0.1 | 0.5 |
| | NEG | <0.05 | 0.1 | 0.5 |
| | NEG | <0.05 | 0.1 | 0.5 |
| | NEG | <0.05 | 0.1 | 0.5 |
| RABV + Vaccine | POS | <0.05 | <0.05 | NA |
| | POS | <0.05 | <0.05 | NA |
| | POS | <0.05 | <0.05 | NA |
| | NEG | <0.05 | <0.05 | 2.67 |
| | POS | <0.05 | <0.05 | NA |
| RABV + GRFT | POS | <0.05 | <0.05 | NA |
| Low | NEG | <0.05 | 331.4 | 331.4 |
| Dose + Vaccine | NEG | <0.05 | 331.4 | 66.67 |
| | POS | <0.05 | <0.05 | NA |
| | NEG | <0.05 | 331.4 | 1663.7 |
| RABV + GRFT | NEG | <0.05 | 331.4 | 1663.7 |
| High | POS | <0.05 | <0.05 | NA |
| Dose + Vaccine | NEG | <0.05 | 331.4 | 1663.7 |
| | POS | <0.05 | <0.05 | NA |
| | NEG | <0.05 | 66.67 | 1663.7 |
| RABV | POS | <0.05 | <0.05 | NA |
| | POS | <0.05 | <0.05 | NA |
| | POS | <0.05 | <0.05 | NA |
| | POS | <0.05 | <0.05 | NA |
| | POS | <0.05 | <0.05 | NA |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing a Rhabdoviridae virus infection in a mammal comprising administering griffithsin, or a fragment or mutant thereof, to the mammal.

2. (canceled)

3. The method of claim 1, wherein the griffithsin mutant comprises SLTHRKFGGSGGSPFSGLSSIAVRSGSYLDAIIIDGVHHGGSGGNLSPTFTFGSGEYISN X₁TIRSGDYIDNISFX₂TNX₃GRRFGPYGGSGGSANTLSNVKVIQINGX₄XSGDYLDSLD X₆YYX₇QY (SEQ ID NO: 1), wherein X₁ can be M or V, X₂ can be E or Q, X₃ can be M, A, K, V, F, L, I, Q, R, or G, X₄ can be S or R, X₅ can be A or S, X₆ can be I or F, and X₇ can be E or Q provided that the polypeptide does not comprise the amino acid sequence of SEQ ID NO: 2.

4. The method of claim 1, wherein the griffithsin mutant comprises SEQ ID NO: 14.

5. The method of claim 1, wherein griffithsin, or a fragment or mutant thereof, is administered orally, subcutaneously, or as an intra-muscular injection.

6. (canceled)

7. (canceled)

8. The method of claim 1, wherein the method comprises administering griffithsin, or a fragment or mutant thereof, to the mammal in a single dose.

9. The method of claim 1, wherein the method comprises administering griffithsin, or a fragment or mutant thereof, to the mammal in multiple doses.

10. The method of claim 1, wherein the method comprises administering griffithsin, or a fragment or mutant thereof, to the mammal in two to five separate doses with a dosing interval of one to five days.

11. The method of claim 1, wherein the method comprises administering griffithsin, or a fragment or mutant thereof, to the mammal every one to five days for a 30 day duration.

12. The method of claim 1, wherein griffithsin, or a fragment or mutant thereof, is co-administered with an additional therapeutic agent.

13. The method of claim 1, wherein the Rhabdoviridae virus is a Lyssavirus.

14. The method of claim 1, wherein the Rhabdoviridae virus is a rabies virus.

15. The method of claim 12, wherein the additional therapeutic agent is rabies immune globulin (RIG), a rabies vaccine, a humanized monoclonal antibody that binds to an epitope on a rabies virus glycoprotein, an antiviral medication, a single stranded RNA with immunostimulating activity, an antiviral medication, or a combination thereof.

16. The method of claim 15, wherein the monoclonal antibody is CTB011, CTB012, RAB1, CR57, CR4098, or a combination thereof.

17. The method of claim 15, wherein the monoclonal antibody is a mixture of CTB011 and CTB012.

18. The method of claim 15, wherein the single stranded RNA with immunostimulating activity is CV8102.

19. The method of claim 12, wherein the additional therapeutic agent is rabies immunoglobulin (RIG).

20. The method of claim 12, wherein the additional therapeutic agent is a rabies vaccine.

21. The method of claim 12, wherein the additional therapeutic agent is PAV-866.

22. The method of claim 1, wherein griffithsin, or a fragment or mutant thereof, is administered to the mammal before onset of neurological signs of rabies virus infection.

23. The method of claim 1, wherein the mammal is a human.

24-27. (canceled)


SEQUENCE LISTING (14 sequences)

SEQ ID NO: 1 - protein, 121 aa, Artificial Sequence
  synthetic
  MISC_FEATURE (61)..(61): wherein Xaa can be M or V
  MISC_FEATURE (75)..(75): wherein Xaa can be E or Q
  MISC_FEATURE (78)..(78): wherein Xaa can be M, A, K, V, F, L, I, Q, R, or G
  MISC_FEATURE (106)..(106): wherein Xaa can be S or R
  MISC_FEATURE (107)..(107): wherein Xaa can be A or S
  MISC_FEATURE (116)..(116): wherein Xaa can be I or F
  MISC_FEATURE (119)..(119): wherein Xaa can be E or Q
SLTHRKFGGSGGSPFSGLSSIAVRSGSYLDAIIIDGVHHGGSGGNLSPTFTFGSGEYISNXTIRSGDYIDNISFXTNXGRRFGPYGGSGGSANTLSNVKVIQINGXXGDYLDSLDXYYXQY

SEQ ID NO: 2 - protein, 121 aa, Artificial Sequence
  synthetic
SLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 3 - protein, 121 aa, Artificial Sequence
  synthetic
SLTHRKFGGSGGSPFSGLSSIAVRSGSYLDAIIIDGVHHGGSGGNLSPTFTFGSGEYISNVTIRSGDYIDNISFQTNKGRRFGPYGGSGGSANTLSNVKVIQINGSAGDYLDSLDIYYEQY

SEQ ID NO: 4 - protein, 121 aa, Artificial Sequence
  synthetic
SLTHRKFGGSGGSPFSGLSSIAVRSGSYLDAIIIDGVHHGGSGGNLSPTFTFGSGEYISNVTIRSGDYIDNISFQTNKGRRFGPYGGSGGSANTLSNVKVIQINGRAGDYLDSLDIYYEQY

SEQ ID NO: 5 - protein, 121 aa, Artificial Sequence
  synthetic
SLTHRKFGGSGGSPFSGLSSIAVRSGSYLDAIIIDGVHHGGSGGNLSPTFTFGSGEYISNVTIRSGDYIDNISFQTNKGRRFGPYGGSGGSANTLSNVKVIQINGSAGDYLDSLDFYYQQY

SEQ ID NO: 6 - protein, 121 aa, Artificial Sequence
  synthetic
SLTHRKFGGSGGSPFSGLSSIAVRSGSYLDAIIIDGVHHGGSGGNLSPTFTFGSGEYISNMTIRSGDYIDNISFQTNKGRRFGPYGGSGGSANTLSNVKVIQINGRAGDYLDSLDIYYEQY

SEQ ID NO: 7 - protein, 121 aa, Artificial Sequence; identical to SEQ ID NO: 1
  synthetic
  MISC_FEATURE (61)..(61): wherein Xaa is M
  MISC_FEATURE (75)..(75): wherein Xaa is Q
  MISC_FEATURE (78)..(78): wherein Xaa is K, V, or A
  MISC_FEATURE (106)..(106): wherein Xaa is S
  MISC_FEATURE (107)..(107): wherein Xaa is A
  MISC_FEATURE (116)..(116): wherein Xaa is I
  MISC_FEATURE (119)..(119): wherein Xaa is E

SEQ ID NO: 8 - protein, 121 aa, Artificial Sequence; identical to SEQ ID NO: 1
  synthetic
  MISC_FEATURE (61)..(61): wherein Xaa is M
  MISC_FEATURE (75)..(75): wherein Xaa is Q
  MISC_FEATURE (78)..(78): wherein Xaa is K
  MISC_FEATURE (106)..(106): wherein Xaa is S
  MISC_FEATURE (107)..(107): wherein Xaa is A
  MISC_FEATURE (116)..(116): wherein Xaa is I
  MISC_FEATURE (119)..(119): wherein Xaa is Q

SEQ ID NO: 9 - protein, 121 aa, Artificial Sequence; identical to SEQ ID NO: 1
  synthetic
  MISC_FEATURE (61)..(61): wherein Xaa is V
  MISC_FEATURE (75)..(75): wherein Xaa is Q
  MISC_FEATURE (78)..(78): wherein Xaa is K
  MISC_FEATURE (106)..(106): wherein Xaa is S
  MISC_FEATURE (107)..(107): wherein Xaa is A
  MISC_FEATURE (116)..(116): wherein Xaa is I
  MISC_FEATURE (119)..(119): wherein Xaa is Q

SEQ ID NO: 10 - protein, 121 aa, Artificial Sequence; identical to SEQ ID NO: 1
  synthetic
  MISC_FEATURE (61)..(61): wherein Xaa is M
  MISC_FEATURE (75)..(75): wherein Xaa is Q
  MISC_FEATURE (78)..(78): wherein Xaa is K
  MISC_FEATURE (106)..(106): wherein Xaa is S
  MISC_FEATURE (107)..(107): wherein Xaa is A
  MISC_FEATURE (116)..(116): wherein Xaa is F
  MISC_FEATURE (119)..(119): wherein Xaa is Q

SEQ ID NO: 11 - protein, 121 aa, Artificial Sequence
  synthetic
SLTHRKFGGSGGSPFSGLSSIAVRSGSYLDAIIIDGVHHGGSGGNLSPTFTFGSGEYISNMTIRSGDYIDNISFETNAGRRFGPYGGSGGSANTLSNVKVIQINGSAGDYLDSLDIYYEQY

SEQ ID NO: 12 - protein, 121 aa, Artificial Sequence
  synthetic
SLTHRKFGGSGGSPFSGLSSIAVRSGSYLDAIIIDGVHHGGSGGNLSPTFTFGSGEYISNMTIRSGDYIDNISFETNKGRRFGPYGGSGGSANTLSNVKVIQINGSAGDYLDSLDIYYEQY

SEQ ID NO: 13 - protein, 121 aa, Artificial Sequence
  synthetic
SLTHRKFGGSGGSPFSGLSSIAVRSGSYLDAIIIDGVHHGGSGGNLSPTFTFGSGEYISNMTIRSGDYIDNISFETNLGRRFGPYGGSGGSANTLSNVKVIQINGSAGDYLDSLDIYYEQY

SEQ ID NO: 14 - protein, 121 aa, Artificial Sequence
  synthetic
SLTHRKFGGSGGSPFSGLSSIAVRSGSYLDAIIIDGVHHGGSGGNLSPTFTFGSGEYISNMTIRSGDYIDNISFETNQGRRFGPYGGSGGSANTLSNVKVIQINGSAGDYLDSLDIYYEQY